(19) United States
(12) Patent Application Publication
(10) Pub. No.: US 2011/0287442 A1
(43) Pub. Date: Nov. 24, 2011

(54) Tumor Antigen Protein, Gene, or Peptides from Topoisimerase 2 Alpha

(75) Inventor: TAI GYU KIM, SEOUL (KR)

(73) Assignee: CATHOLIC UNIVERSITY INDUSTRY ACADEMIC COOPERATION FOUNDATION, SEOUL (KR)

(21) Appl. No.: 12/907,863

(22) Filed: Oct. 19, 2010

(30) Foreign Application Priority Data
May 20, 2010 (KR) 1020100047591

(51) Int. Cl. G01N 33/53; C07H 21/02; C12N 5/10; C07H 21/04; C12Q 1/02; C12N 9/90

(52) U.S. Cl. 435/7.1; 435/29; 435/233; 435/325; 536/23.2

(57) ABSTRACT

Provided is a tumor antigen protein, gene, or peptides derived from topoisomerase 2 alpha. As it is discovered that topoisomerase 2 alpha binds to an MHC class I or II antigen, thereby forming a complex, and the complex is recognized by cytotoxic T lymphocytes, the tumor antigen can be used in tumor immunotherapy.

### CROSS-REFERENCE TO RELATED APPLICATION

This application claims priority to and the benefit of Korean Patent Application No. 2010-0047591, filed May 20, 2010, the disclosure of which is incorporated herein by reference in its entirety.

### BACKGROUND

1. Field of the Invention

The present invention relates to a tumor antigen protein, gene, or peptides derived from topoisomerase 2 alpha (Top2α or TopIIα), and more particularly, to a pharmaceutical use of a tumor antigen protein, gene or peptides derived from Top2α forming a complex with an MHC class I or II antigen, the complex being recognized by T lymphocytes.

2. Discussion of Related Art

Recently, finding a fundamental solution to reduce or prevent death due to cancer has been difficult because of side effects of conventional cancer therapies, and acquisition of a tolerance caused by disseminated tumor cells and genetic instability thereof. For these reasons, development of anti-tumor vaccines based on an immune surveillance system is attracting attention. Until now, various vaccines including synthetic peptide vaccines, genetic vaccines, protein vaccines, etc. have been studied, and among these, a dendritic cell-based vaccine is considered the most effective. Dendritic cells (DCs) are the most potent and important antigen-presenting cells in vivo, and thus are highly applicable to most immune response-involving diseases as well as anti-tumor vaccines.

Meanwhile, although it is important to identify tumor antigens recognized by cytotoxic T lymphocytes for successful cancer immunotherapy, a limited number of tumor antigens capable of being used in immunotherapy have been known so far.

An important immunotherapeutic strategy for tumors includes using DCs, which are the most potent antigen-presenting cells for effectively activating immune responses, and effectively presenting a tumor antigen to T lymphocytes by effectively delivering the tumor antigen to the DC.

Immunological treatment of cancer may be provided by killing a cancer cell by potent cancer-specific cytotoxic T lymphocytes induced when a cancer-specific antigen is sensitized to a cell having an antigen-presenting ability and thus the cytotoxic T lymphocytes are activated. The cell having the antigen-presenting ability is very useful in anti-cancer immunotherapy because the antigen-presenting cell forms a complex with a major histocompatibility complex (MHC) recognizing and taking an antigen, thereby presenting the antigen to the T lymphocytes, co-stimulatory factors sufficiently expressed in the cell having the antigen-presenting ability play an important role in the reaction of a T lymphocyte receptor with the antigen-MHC complex, and the antigen-presenting cell secretes various cytokines associated with the differentiation, growth or influx of the T lymphocytes to regulate the activation of the immune responses.

Top2 is an enzyme resolving the topological problem of DNA by breaking and re-sealing DNA and playing an important role in cell viability and division. There are two subclasses of Top2: Top2α and Top2β. Top2β is expressed in a normal cell, but Top2α is highly expressed in a proliferating cell. For example, Top2α is expressed in various cancer tissues including breast cancer, ovarian cancer, and stomach cancer tissues in a higher level than in normal cells.

If Top2α induces immune responses specific to tumor cells, it can be used as a target of immunotherapy specifically removing tumor cells. However, very little is known of Top2α.

### SUMMARY OF THE INVENTION

The present invention is directed to investigation of a specific immune response of Top2α in tumor cells to provide a use of Top2α as a tumor antigen, and an immunotherapeutic use thereof.

In one aspect, there is provided a tumor antigen including: protein fragments, peptides, or derivatives having functionally equivalent characteristics thereto derived from C-terminal of Top2α, and recognized by cytotoxic T lymphocytes by binding to an MHC class I or II antigen.

In another aspect, there is provided a composition for preventing or treating tumors including a tumor antigen or a gene encoding the same.

In still another aspect, there is provided an antigen-presenting cell presenting a complex between an MHC class I or II antigen and a tumor antigen on a surface of a cell having an antigen-presenting ability.

In yet another aspect, there is provided an antigen-presenting cell prepared by introducing a gene encoding a tumor antigen into a cell having an antigen-presenting ability, and presenting a complex between an MHC class I or II antigen and the tumor antigen.

In yet another aspect, there is provided a composition for preventing or treating tumors including an antigen-presenting cell.

In yet another aspect, there is provided a cytotoxic T lymphocyte specifically recognizing a complex between an MHC class I or II antigen and a tumor antigen, which is presented on an antigen-presenting cell.

In yet another aspect, there is provided a method for preparing a cytotoxic T lymphocyte including stimulating an isolated lymphocyte with a tumor antigen.

In yet another aspect, there is provided a composition for preventing or treating tumors including cytotoxic T lymphocytes.

In yet another aspect, there is provided a composition for detecting cellular immune responses including at least one selected from the group consisting of Top2α protein, a tumor antigen, and an antibody specifically binding to the tumor antigen.

### BRIEF DESCRIPTION OF THE DRAWINGS

The above and other objects, features and advantages of the present invention will become more apparent to those of ordinary skill in the art by describing in detail exemplary embodiments thereof with reference to the attached drawings, in which:

FIG. 1 shows comparison of expression of Top2α in various tumor cell lines and normal mouse tissue;

FIG. 2 shows specific T cell response between Top2α and survivin in mice inoculated with MC38 cell line lysate-pulsed DCs;

FIG. 3 shows induction of Top2α-specific immune responses by inoculation of Top2αC RNA/DC;

FIG. 4 shows activity of cytotoxic T cells with respect to Top2α-expressing cells;

FIGS. 5 to 7 show anti-tumor effects mediated by inoculation of Top2αC RNA/DC in mouse tumor models;

FIG. 8 shows effects of the absence of CD4 and CD8 T cells in MC38 tumor models inoculated with Top2αC RNA/DC; and

FIG. 9 shows production of IFN-γ by peptides predicted from mice inoculated with Top2αC RNA/DC and anti-tumor effects mediated by p1327 peptides-pulsed DCs.

### DETAILED DESCRIPTION OF EXEMPLARY EMBODIMENTS

Hereinafter, the present invention will be described with reference to examples and comparative examples in detail. However, the present invention is not limited to these examples.

The present invention relates to a tumor antigen including protein fragments, peptides, or derivatives having functionally equivalent characteristics thereto derived from C-terminal of Top2α, and recognized by cytotoxic T lymphocytes by binding to an MHC class I or II antigen.

The inventors found that Top2α or a mutant protein thereof binds to an MHC class I or II antigen by decomposition in a cell, thereby forming a complex, and the complex can produce peptide fragments which can be recognized by T cells. Therefore, the present invention provides a tumor antigen which can induce a tumor-specific immune response from Top2α or a mutant protein thereof by forming the complex with an MHC class I or II antigen.

The tumor antigen may include protein fragments, peptides, or derivatives having functionally equivalent characteristics thereto derived from C-terminal of Top2α.

The protein fragments, peptides or derivatives having functionally equivalent characteristics thereto derived from the C-terminal of Top2α may be derived from, but are not limited to, humans or mice.

In detail, the protein fragments derived from the C-terminal may use, but are not limited to, an amino acid sequence set forth in SEQ ID NO: 2 or 4.

The protein fragments derived from the C-terminal may use a mutated sequence in which at least one amino acid residue of the amino acid sequence set forth in SEQ. ID. NO: 2 or 4 is substituted, deleted or added.

Herein, the term “a mutated sequence in which at least one amino acid residue of the amino acid sequence is substituted, deleted or added” refers to an amino acid sequence of artificially-modified protein or protein having functionally equivalent characteristics acquired by polymorphism, mutation or modification generally found in protein-coding DNA. DNA encoding such a mutant protein may be prepared by site-directed mutagenesis or PCR. Here, the number of amino acid residues substituted, deleted or added refers to the number of amino acid residues substituted, deleted or added by a known method such as the site-directed mutagenesis.

Herein, the term “peptides which can bind to an MHC class I or II antigen by decomposition in a cell and be recognized by T cells in the binding state” refers to a part of peptides composed of a part of an amino acid sequence of a tumor antigen protein or its mutant protein is capable of binding to an MHC class I or II antigen. When the peptide fragment binds to the MHC class I or II antigen and thus are presented on a surface of a cell, a T cell may be specifically bound to the complex between the peptide fragment and the MHC class I or II antigen, thereby transferring a signal to the T cell. Further, the “bond or binding” used herein refers to a non-covalent bond. In detail, the peptide may use a sequence set forth in SEQ ID NO: 5, but the present invention is not limited thereto.

Herein, the term “derivatives having functionally equivalent characteristics” refers to derivatives having functionally equivalent characteristics to protein fragments derived from the C-terminal of Top2α or peptides. In detail, the derivatives include those in which some amino acid residues of the peptides are substituted, deleted or added, and those in which some amino acid residues of the peptides are substituted, deleted or added, and have a modified amino or carboxyl group.

As the derivatives in which some of the amino acid residues of the peptides are substituted, deleted or added, preferably, the derivatives of the tumor antigen peptides in which epitope regions involved in binding to CTL are conserved and amino acid residues involved in binding with an MHC class I or II antigen are substituted, deleted or added may be used, and more preferably, the derivatives of the peptides in which only one amino acid residue is substituted, may be used (refer to Immunol. 84:298-303, 1995).

Such derivatives can be easily synthesized using a commercially available peptide synthesizer, and the binding affinity of synthesized derivatives to MHC class I antigens may be easily measured by competitive inhibition assay using a cell-free system between the derivatives and radiolabeled standard peptide for binding to an MHC class I or II antigen (R. T. Kubo et al., J. Immunol., 152:3913, 1994). Thus, by subjecting various peptide derivatives to such assay, peptide derivatives having CTL-inducing activity can be easily selected. Since the peptide derivatives thus selected can bind to an MHC class I or II antigen more strongly while retaining their binding ability to CTL, they can be used as more efficient tumor antigen peptides.

A modifying group of an amino group may be an acyl group. Particularly, examples of modifying groups of an amino group may include an alkanoyl group having 1 to 6 carbon atoms, an alkanoyl group having 1 to 6 carbon atoms substituted with a phenyl group, a carbonyl group substituted with a cycloalkyl group having 5 to 7 carbon atoms, an alkylsulfonyl group having 1 to 6 carbon atoms, a phenylsulfonyl group, and the like.

Modifying groups of a carboxyl group may include, for example, ester and amide groups. Specific examples of such ester groups may include an alkyl ester group having 1 to 6 carbon atoms, an alkyl ester group having 1 to 6 carbon atoms substituted with a phenyl group, and a cycloalkyl ester group having 5 to 7 carbon atoms, and specific examples of such amide groups may be an amide group, an amide group substituted with one or two alkyl groups having 1 to 6 carbon atoms, an amide group substituted with one or two alkyl groups having 1 to 6 carbon atoms substituted with a phenyl group, and an amide group forming 5 to 7-membered azacycloalkane containing a nitrogen atom of the amide group.

The present invention also relates to a composition for preventing or treating tumors including a tumor antigen of the present invention or a gene encoding the same.

The tumor may be, but is not particularly limited to, glioma, colorectal cancer, or malignant melanoma, expressing Top2α.

A medicine containing tumor antigen protein fragments, peptides, or derivatives having functionally equivalent characteristics thereto as active ingredients may be administered to tumor patients to treat or prevent a tumor. As the tumor antigen binds to an MHC class I or II antigen in a cell, and is thus highly presented on a surface of the cell, tumor-specific CTL is effectively proliferated in vivo, resulting in treatment or prevention of the tumor.

The tumor antigen may use independently or at least two of protein fragments, peptides, and derivatives functionally equivalent characteristics thereto, and the medicine containing them as active ingredients may be administered with an adjuvant, or formulated in a particle.

The gene encoding a tumor antigen of the present invention acts as a tumor-specific antigen, and may be involved in preventing or treating tumors as the tumor antigen produced by expression of the gene reinforces a tumor-specific immune response.

The medicine containing the gene of the present invention as an active ingredient can treat or prevent a tumor. When the gene of the present invention is administered to a tumor patient, a tumor antigen protein in a cell is highly expressed, and tumor antigen peptides bind to an MHC class I or II antigen to be highly presented on a surface of the cell, thereby effectively proliferating tumor-specific CTL in vivo.

The gene may be administered in the form of DNA or RNA.

To administer DNA to be introduced into a cell, a method using a viral vector, but not limited thereto, may be used. Examples of the methods using the viral vectors may include methods in which DNA of the present invention is incorporated into DNA or RNA virus such as retrovirus, adenovirus, adeno-associated virus, herpesvirus, vaccinia virus, poxvirus, poliovirus, or Sindbis virus, and introduced into cells. Among these methods, those using retrovirus, adenovirus, adeno-associated virus, or vaccinia virus are particularly preferred.

As a method of administering and introducing RNA into cells, electroporation may be used, but the present invention is not limited thereto.

To actually apply the gene of the present invention to a medicine, an in vivo method for directly introducing a gene into the body, or an ex vivo method including collecting certain cells from a human body, introducing a gene into the cells ex situ, and reintroducing the cells into the body may be used. The in vivo method is preferred.

In the case of the in vivo method, the gene may be administered through any appropriate route depending on the diseases and symptoms to be treated, and other factors. For example, it may be administered by intravenous, intraarterial, subcutaneous, Intramuscular, or intradermal injection. The administration technique may be single or multiple administrations. A composition for administration may be formulated in various forms such as a solution, typically, in a carrier-added injection. When the gene is contained in liposomes or cell membrane-fused liposomes, such medicines may be in the form of liposome formulations such as a suspension, a frozen drug, or a centrifugally-concentrated frozen drug.

The composition of the present invention may include a pharmaceutically available carrier and/or diluent. The carrier and diluent alone may not make a harmful reaction with respect to an object to which the composition is administered. In detail, the carrier may be protein, polysaccharide, polylactic acid, polyglycolic acid, polymeric amino acid, amino acid copolymer, liposome, and an inactive virus particle, which may be suitably selected according to the necessity of one of ordinary skill in the art. The diluent may be water, saline, glycerol or ethanol. The composition may further include a wetting agent, an emulsifier, a pH buffer, etc.

Suitable injectable formulations may be sterile solutions or dispersions for instantly preparing sterile injectable solutions or dispersions and sterile powder. These must be stable under preparing conditions, and retained against contamination with microorganisms such as bacteria or fungi. The contamination with microorganisms may be prevented using various antibacterial and antifungal agents, for example, paraben, chlorobutanol, phenol, sorbic acid, thimerosal, and the like. It may also be desirable to include isotonic agents, such as sugars or sodium chloride in the injectable formulations. In addition, prolonged absorption of the injection may be brought about by the inclusion of agents which delay absorption such as aluminum monostearate or gelatin.

Although the amount of the gene in such a composition of the present invention may vary depending on, for example, the disease to be treated, the age and weight of a particular patient, and the like, it is usually preferred to administer 0.0001 to 100 mg, more preferably 0.001 to 10 mg, of the gene of the present invention every several days to several months.

It is important to establish an administration method to effectively induce CTL specific to the body of the patient when a tumor antigen or a gene encoding the tumor antigen of the present invention is used to treat a tumor. As an example, the present invention provides an antigen-presenting cell in which a complex between the MHC class I or II antigen and the tumor antigen of the present invention is presented on the surface of the cell having an antigen-presenting ability.

The antigen-presenting cell of the present invention has a tumor antigen loaded, thereby inducing a tumor-specific immune response.

The term “loading or pulsing” indicates that antigen-presenting cells (APCs) such as DCs display antigens captured, decomposed, and loaded to an MHC molecule to the surfaces of the cells. As a result, the antigen-loaded cells can induce potent activation of antigen-specific T lymphocytes.

The antigen-presenting cell expresses an MHC class I or II antigen capable of presenting the tumor antigen of the present invention on the surface of the cell. Examples of the cells may include, but are not limited to, dendritic cells, monocytes, CD4 T cells, B cells, and gamma delta T cells. The CD4 T cells, B cells, or gamma delta T cells may be in a naive, activation, or expansion state.

The cells having an antigen-presenting ability are separated from bone marrow cells to prepare the antigen-presenting cells of the present invention from the cells having an antigen-presenting ability, and a pulse is applied to the tumor antigen of the present invention outside the body to form a complex between the MHC class I or II antigen and the tumor antigen.

In the case of using the dendritic cells, the antigen-presenting cells of the present invention may be prepared by separating lymphocytes from bone marrow cells by a Ficoll method, removing non-adherent cells, incubating adhesive cells in the presence of GM-CSF and IL-4 to induce the dendritic cells, incubating the dendritic cells as the tumor antigens of the present invention, and stimulating the cells with pulse.

The present invention also provides an antigen-presenting cell presenting the complex between the MHC class I or II antigen and the tumor antigen, which is prepared by introducing a gene encoding the tumor antigen of the present invention into a cell having an antigen-presenting ability.

The gene may use a base sequence set forth in SEQ ID NO: 1 or 3, but the present invention is not limited thereto.

The antigen-presenting cell of the present invention may be prepared by: separating a lymphocyte from a bone marrow cell; loading a gene encoding a protein fragment(s), peptides, or derivatives having functionally equivalent characteristics thereto derived from C-terminal of Top2α to the cell; and maturing the cell.

In detail, the antigen-presenting cell may be prepared by: (a) inducing a DC from a bone marrow cell under GM-CSF and IL-4; (b) loading C-terminal RNA of Top2α to the DC; and (c) adding LPS as a maturation-inducing factor to the C-terminal RNA of Top2α-loaded DC to mature the DC.

The C-terminal RNA of Top2α may be prepared by mixing a T7 RNA polymerase with a C-terminal RNA of Top2α-loaded vector and performing in vitro transcription. Here, the vector may be a pcDNA3.1 TOPO vector, but the present invention is not limited thereto.

In the preparation method of the present invention, the loading of the C-terminal RNA of Top2α in step (b) may be performed by electroporation, and step (c) may be performed by adding GM-CSF, IL-4, and LPS at concentrations of 10 to 20 ng/ml, 10 to 20 ng/ml and 1 μg/ml, respectively, and incubating the vectors for 12 hours to 2 days, preferably, 1 day.

The present invention relates to a composition for preventing or treating tumors including the antigen-presenting cells of the present invention.

An immune response may be induced by vaccination with the antigen-presenting cells of the present invention, which is particularly useful in inducing a reaction of cytotoxic T lymphocytes with respect to antigens. The immune response may be a specific (T lymphocyte) and/or non-specific immune response. In detail, CD8⁺ and CD4⁺ T lymphocytes are involved in the suppression of tumor growth by vaccination with the antigen-presenting cells of the present invention.

The pharmaceutical composition for preventing or treating tumors containing the antigen-presenting cell of the present invention as an active ingredient may be administered with other immunomodulators.

The pharmaceutical composition of the present invention may further include at least one pharmaceutically available carrier and/or diluent, in addition to the antigen-presenting cell. Examples of the carriers and/or diluents are described above.

As described above, another aspect of the present invention provides a method of inducing or stimulating an immune response against an antigen in a human, particularly, a cancer patient. The method includes administrating an effective dose of the vaccine composition or antigen-presenting cells described above to the cancer patient.

Still another aspect of the present invention provides a use of the antigen-presenting cell of the present invention to treat and/or prevent a disease. Examples of diseases capable of being treated by the method of the present invention include incurable cancers such as glioma, colorectal cancer, and malignant melanoma.

Thus, yet another aspect of the present invention provides a method of treating incurable cancers such as glioma, colorectal cancer, and malignant melanoma by reinforcing a cancer-specific immune response using the antigen-presenting cell vaccine. Here, the administration of the composition induces or otherwise stimulates an immune response suppressing, stopping, prolonging, or preventing occurrence or procession of a disease state.

The term “effective dose” refers to an amount necessary to at least partially ensure a desired immune response, or delay or completely stop the occurrence or progression of a specific disease to be treated. The dose may vary depending on various factors such as health and physical states of an object to be treated, a taxological group of the object to be treated, ability of an immune system of an object synthesizing an antibody, a desired level of protection, vaccination formulations, evaluation of medical conditions, and other related factors. The dose is expected to be included in a relatively wide range to be measured by a common attempt. For example, to treat a kidney cancer, 2×10⁶ to 2×10⁷ cells/dosage may be injected for a 60 kg adult.

The present invention also relates to a cytotoxic T lymphocyte specifically recognizing a complex between an MHC class I or II antigen and a tumor antigen of the present invention, presented on the antigen-presenting cell of the present invention, and a method of preparing the same by stimulating a isolated lymphocyte with the tumor antigen.

The cytotoxic T lymphocyte of the present invention may be a CD4 or a CD8 T cell, which induces a tumor-specific immune response by recognizing the complex between the tumor antigen and the MHC class I or II antigen.

The pharmaceutical composition of the present invention may include physiological saline, phosphate buffered saline (PBS), and a medium to stably maintain the cytotoxic T lymphocyte. The pharmaceutical composition may be administered intravenously, subcutaneously or percutaneously. To treat a tumor, as the composition containing the cytotoxic T lymphocyte, as an active ingredient, is re-injected into the body of a patient, the specific cytotoxic T lymphocyte for tumor treatment is effectively introduced to the Top2α-positive patient. It is natural that there is compatibility in MHC types between the patient and peptides to be used.

The present invention also relates to a composition for measuring a cellular immune response including at least one selected from Top2α protein, a tumor antigen of the present invention, and an antibody specifically binding to the tumor antigen.

The measurement of the cellular immune response may be performed to diagnose a tumor.

The cellular immune response may be measured using a cell specifically binding to the Top2α protein, the tumor antigen of the present invention, and the antibody specifically binding to the tumor antigen. For example, the method of measuring the cellular immune response may be a method of detecting a tumor antigen protein from a tumor tissue sample, or a method of detecting the presence of a tumor antigen protein in blood or tissue or a cell with respect to the tumor antigen protein. The detection method may be suitably selected from immunohistochemistry, immunoblotting, radioimmunoassay (RIA), enzyme-linked immunosorbent assay (ELISA), and fluorescent or luminescent immunoassay. As a tumor antigen protein is detected using a cell, early detection of tumors and diagnosis of reoccurrence are possible, and tumor patients to whom medicines including tumor antigen proteins, tumor antigen peptides or genes encoding the same can be applied may be selected.

The tumors may be diagnosed by immunological diagnostic methods using antibodies specifically binding to tumor antigen proteins, tumor antigen peptides, or derivatives thereof. For example, the immunological diagnosis may be performed by labeling a suitable amount of antibodies, and detecting the presence of antigens in a sample (such as blood or a tumor tissue) obtained from a patient suspected to have a tumor by the labeled antibodies.

Further, antigen-specific cytotoxic T lymphocytes may be detected using the complex between the tumor antigen and the MHC antigen. Specifically, a tetramer between a fluorescently-labeled MHC antigen and tumor antigen peptides may be prepared, and the antigen-specific cytotoxic T lymphocytes from peripheral blood lymphocytes obtained from a patient suspected to have a tumor may be quantitatively analyzed using flow cytometry.

Hereinafter, the present invention will be described in further detail with respect to examples according to the present invention and comparative examples not according to the present invention, but the scope of the present invention is not limited by the following examples.

### Preliminary Experimental Example 1

### Experimental Animals and Cancer Cell Culture

6 to 8-week-old female C57BL/6 mice (H-2^{b}) were purchased from Orient Bio Inc. (Gapyeong, Gyeonggi-do, Korea).

GL26 (H-2^{b}; glioma), MC-38 and MC-38-cea2 (H-2^{b}; colorectal cancer), B16F10 (H-2^{b}; malignant melanoma), and CT26 (H-2^{d}; colorectal cancer) were cultured with 10% fetal bovine serum (FBS; Gibco, Grand Island, N.Y., USA), 2 mM glutamine, 100 U/ml penicillin, and 100 μg/ml streptomycin in complete Dulbecco's modified Eagle media (DMEM).

EL4 (H-2^{b}; lymphoma) and YAC-1 (H-2a; lymphoma) were cultured in complete RPMI-1640 (Cambrex) media supplemented with 10% heat-inactivated fetal bovine serum, 2 mM glutamine, 100 U/ml penicillin, and 100 μg/ml streptomycin.

GL26 was provided by Dr. Yu (Cedars Sinai Medical Center, Los Angeles, Calif., USA), MC-38-cea2 expressing human CEA was provided by Dr. Schlom (Division of Tumor Immunology and Biology, NIH, Bethesda, Md., USA), and MC-38, EL4, B16F10, CT26 and YAC-1 were purchased from American Type Culture Collection (ATCC, Manassas, Va., USA).

### Preliminary Experimental Example 2

### Statistical Analysis

Test results were expressed as the means±the standard deviation (SD) or standard error (SE). Statistical analysis was carried out by Student's t-test, excluding the survival data. The survival data were analyzed using the Kaplan-Meier test, and compared with other groups using a log-rank test. A p value of <0.05 was considered to be a statistically significant difference.

### Example 1

### Preparation of Top2αC RNA-Loaded DC-Based Vaccine

<1-1> Cloning of Top2αC

Here, a C-terminus of mouse Top2α was used, which consists of amino acid residues 1263-1528 (Top2αC). Reverse-transcribed mRNA for mouse Top2αC isolated from GL26 was used as a template for polymerase chain reaction (PCR) amplification. PCR conditions were as follows: 2 minutes at 94° C.; followed by 30 cycles of 15 seconds at 94° C. and 1 minute at 72° C.; and 10 minutes at 72° C.

Primers for Top2αC were 5′-CTGAGGATGGTGCAGAAGAAAGAGCCGGT-3′ (forward primer) and 5′-TGCCTCAGAAGAGGTCGTCATCGTCATCGAA-3′ (reverse primer). A PCR product was inserted into pcDNA3.1 TOPO vector (Invitrogen, Grand island, NY, USA). A cloned gene was identified through base sequence analysis.

<1-2> Generation of Bone Marrow-Derived DCs

To generate DCs, after bone marrow cells were harvested from bone marrows of femurs and tibias of the 6 to 8-week-old female C57BL/6 mice, erythrocytes were removed using hypotonic buffer (9.84 g/L NH₄Cl, 1 g/L KHCO₃, and 0.1 mM EDTA). The cells were washed twice with a serum-free RPMI-1640 medium and cultured at 5×10⁶ cells/well in complete RPMI-1640 containing 20 ng/ml of murine granulocyte-macrophage colony-stimulating factors (GM-CSF) (R&D Systems, Minneapolis, Minn., USA) and 20 ng/ml of recombinant murine interleukin-4 (IL-4, R&D System). After 48 hours, non-adherent cells were removed, and a fresh complete medium containing GM-CSF and IL-4 was added. On day 6 after the culture, non-adherent and loosely-adherent cells (DCs) were harvested, and subjected to RNA electroporation.

<1-3> RNA Electroporation into DCs

Top2αC/pcDNA3.1 TOPO plasmid was digested with SmaI, and subjected to in vitro transcription (IVT) together with T7 RNA polymerase (according to the manufacturer's instructions using Ambion mMESSAGE mMACHINE T7 Ultra kit; Austin, Tex., USA). The preparation of IVT RNA was identified by agarose gel electrophoresis, and RNA concentration was measured by spectrophotometer. Aliquots of RNA samples were stored at −70° C. 6 days after the culture, dendritic cells were suspended in an Opti-MEM medium at a concentration of 2.5×10⁷ cells/ml, 200 μl of the cell suspension was put into a 2 mm cuvette, and then 20 μg of RNA was added thereto. The cell suspension was placed on an ElectroSquarePorator (ECM 830, BTX, San Diego, Calif., USA), and a current of 300 V was applied for 500 μs. The cells were instantly removed from the cuvette, and put into a complete medium containing GM-CSF, IL-4, and LPS (1 μg/ml: Sigma, Saint Louis, Mo., USA) to be matured for 24 hours. Transfectional efficiency of the dendritic cells by electroporation was assessed with GFP RNA by FACS analysis.

<1-4> Preparation of Tumor Lysates

MC38, MC-38-cea2 or B16F10 cells were resuspended at 1×10⁷ cells/ml in PBS. The cell suspension was frozen in liquid nitrogen and thawed in a water bath at 37° C. The freeze-thaw cycle was repeated four times in rapid succession. Large particles were removed by centrifugation at 600 rpm for 10 minutes. The supernatant was passed through a 0.2-μm-pore-size filter, and an aliquot was stored at −70° C. Protein concentration of the lysate was measured using the bicinchoninic acid (BCA) assay (Pierce, Rockford, Ill., USA). Dendritic cells were cultured with 100 μg/ml of MC-38 tumor lysate for 16 to 18 hours to load the MC-38 tumor lysate.

### Experimental Example 1

### Western Blotting

Mouse tissues were minced with a scalpel, cut into small pieces, and suspended in an RIPA buffer containing 1 mM DTT, 1 mM phenylmethanesulfonyl fluoride, 10 μg/ml aporotinin, and 5 μg/ml leupeptin. The suspension was then homogenized using the Precellys24 lyser (Bertin Technologies, Cedex, France). Tumor cells were lysated in the same buffer as described above. The protein concentration was measured using the BCA assay (Pierce, Rockford, Ill., USA). Total proteins (50 μg) were separated on 8% polyacrylamide gel, blotted onto nitrocellulose membranes, and incubated with anti-Top2α antibodies (Epitomics, Burlingame, Calif., USA). Bands were visualized by enhanced chemiluminescence (ECL) staining (Amersham Pharmacia, Freiburg, Germany).

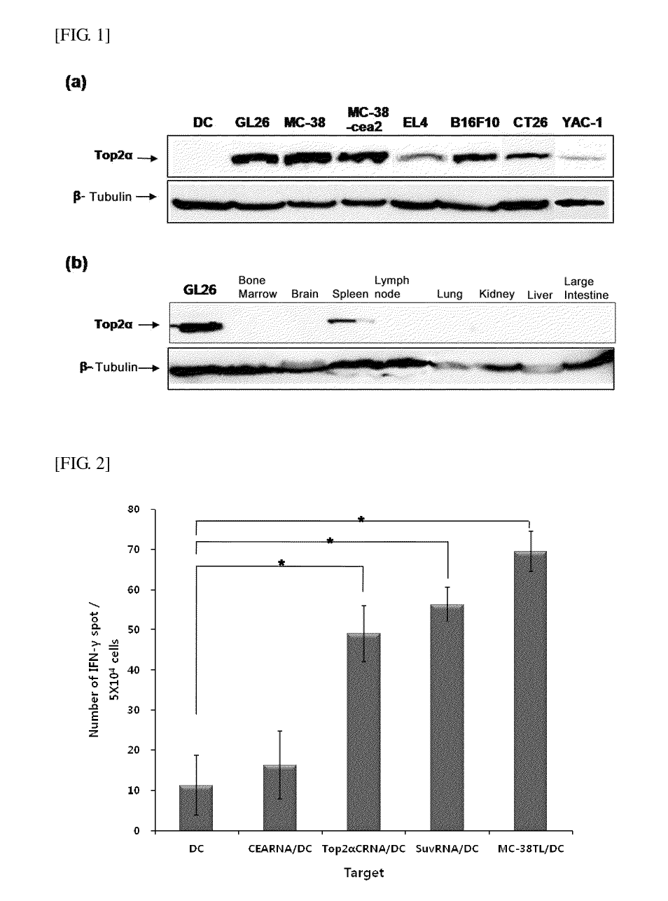

Expression of Top2α in murine tumor cell lines was evaluated by western blotting using the anti-Top2α antibodies, from which it can be confirmed, as shown in FIG. 1, that the Top2α proteins were expressed in various murine tumor cell lines including MC-38 and GL26.

While expression of Top2α in normal murine tissues was evaluated from bone marrow, brain, spleen, lymph node, lung, kidney, liver and large intestine (or colon), as shown in FIG. 1B, it was not found in tissues other than the spleen.

### Experimental Example 2

### Separation of CD4⁺ and CD8⁺ T Lymphocytes

To measure CD4+ and CD8+ T lymphocyte immune response, splenocytes were reacted with magnetic beads conjugated to monoclonal antibodies specific to CD4 or CD8 (MACS; Miltenyi Biotec, Bergisch Gladbach, Germany) for 15 minutes at 4° C. After incubation, the cells were washed with PBS containing 2 mM EDTA, and passed through a MACS magnetic separation column. The purity of each T lymphocyte after the separation was >90%, determined by fluorescent-activated cell sorer (FACS) analysis.

### Experimental Example 3

### Enzyme-Linked Immunospot (ELISPOT) Assay

An ELISPOT kit was purchased from BD Bioscience (Qume Drive, San Jose, Calif., USA), and the ELISPOT assay was performed according to the manufacturer's instructions.

The splenocytes (5×10⁴ cells/well) were seeded into a 96-well plate coated with anti-mouse IFN-γ antibodies. Top2αC RNA/DC, MC-38TL/DC, SuvRNA/DC and DCs were added as target cells. The plates were incubated for 20 hours at 37° C. The cells were removed, and the plate was washed three times with a washing buffer. Biotinylated anti-mouse IFN-γ antibodies were added to the cells, and the plates were incubated for 2 hours at room temperature, and washed three times. Streptavidin-hoseradish peroxidase was added to each well, and the plates were incubated for 1 hour at room temperature. After washing the plates, chromogenic substrate [3-amino-9-ethyl carbazole (AEC)] was added to each well. After spots developed, the reaction was quenched with distilled water, and the plates were dried in a dark room overnight. The number of spots corresponding to the IFN-γ secreting cells was determined with an AID-ELISPOT reader (Strassberg, Germany).

To examine whether the immune responses of mice vaccinated with MC-38 tumor lysate-loaded DCs (MC-38TL/DCs) were induced against Top2α, the number of IFN-γ secreting cells was determined by the ELISPOT assay. The Top2α used herein was C-terminus, consisting of amino acid residues 1263-1528. Since it was found that the MC-38 expressed some tumor antigens including survivin and Top2α, C57BL/6 mice were vaccinated with the MC-38TL/DCs. In 1 week after the second vaccination, the splenocytes were harvested and activated as various target cells.

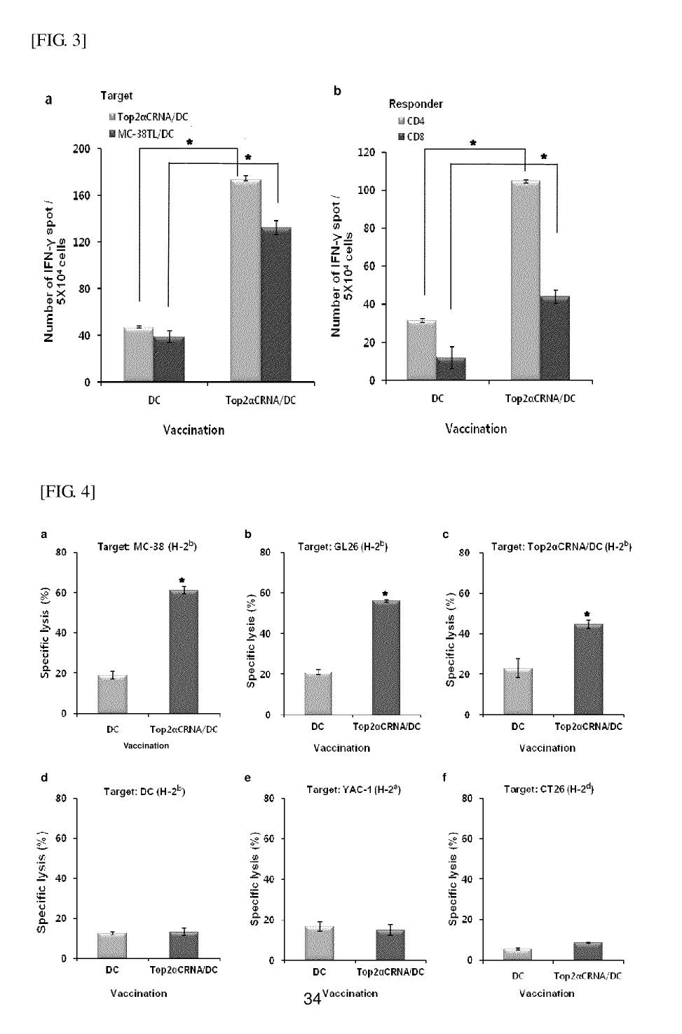

When the cells were stimulated with Top2αC RNA/DCs, compared to when cells were stimulated with the DCs on which the antigens were not loaded, the number of the IFN-γ secreting cells was significantly increased. When the MC-38TL/DCs and survivin were stimulated with RNA-transfected DCs (SurRNA/DCs), the number of the IFN-γ secreting cells was detected in a higher level (see FIG. 2). CEA-pulsed DCs, irrelevant tumor antigens, and unfulsed DCs were used as Top2α—negative cells.

The results demonstrated that the immune responses of the mice for some tumor antigens were induced by vaccination with MC-38TL/DCs, and Top2αC RNA/DCs were capable of presenting a Top2αC epitope (antigen-determining group).

To detect the immune responses induced by the Top2αC RNA/DCs in vivo, after the third vaccination with Top2αC RNA, Top2α-specific IFN-γ secreting T lymphocytes were assessed by quantitative analysis using ELISPOT assay. When the cells were activated by Top2αC RNA/DCs, the frequency of IFN-γ secreting cells in splenocytes, CD4⁺ and CD8⁺ T lymphocytes was significantly higher in the mice vaccinated with Top2αC RNA/DCs, as compared to the mice vaccinated with DCs. The frequency of Top2αC-specific CD4⁺ T lymphocytes was higher than that of the CD8⁺ T lymphocytes.

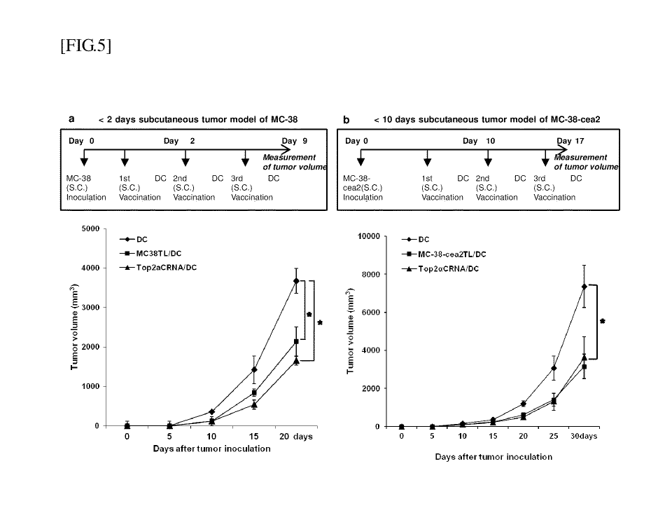

When the cells were activated with DC in which the tumor lysates of the tumor cell lines expressing Top2α, MC38, were loaded, the IFN-γ secreting cells were observed with a high frequency in the splenocytes (FIG. 3A).

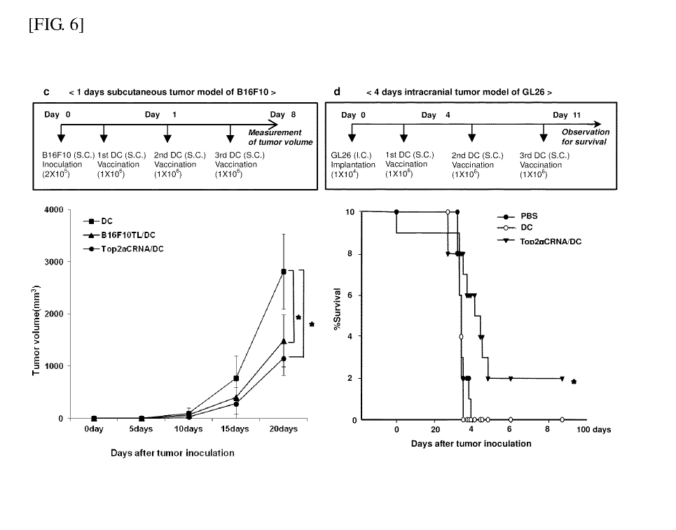

The cytotoxicity of Top2αC-specific cytotoxic T lymphocyte (CTL) was evaluated in various target cells. T lymphocytes from the C57BL/6 mice vaccinated with Top2αC RNA/DCs showed killing activity against MC-38 (H-2^{b}), GL26 (H-2^{b}) and Top2αC RNA/DC(H-2^{b}), as Top2α-expressing cells, at an E/T ratio of 40, but, showed very low cell lysis with respect to the un-pulsed DC as Top2α-unexpressing cells (FIGS. 4A to 4D).

YAC-1 (H-2^{a}) exhibited low cell lysis, which indicated that the killing activity against the target cell was not influenced by NK cells (FIG. 4E). CT26, Top2α-expressing cells with an H-2^{d} haplotype, were not able to be lysed because the Top2α-specific T lymphocytes from C57BL/6 mice were restricted to H-2^{b} MHC class I molecules.

The result demonstrates that Top2α-specific immune responses were successfully induced in vivo by the vaccination with Top2αC RNA/DCs.

### Experimental Example 4

### Cytotoxicity Analysis

A standard ⁵¹Cr-releasing analysis was performed. Splenocytes were extracted from mice, and reactivated in vitro with 4% paraformaldehyde-prefixed MC-38 cells for five days, thereby being used as effector cells. MC-38, GL26, Top2αC RNA/DC, DC, YAC-1 and CT26 labeled with [⁵¹Cr]-sodium chromate (100 mCi/1×10⁶ cells) for 1 hour at 37° C. in the presence of 5% CO₂ were used as target cells. 100 μl of a supernatant of each well was collected, and radioactivity was counted with a gamma counter.

Specific Lysis=100×[(Experiment Release−spontaneous release)/(maximum release−spontaneous release)]

The spontaneous release and the maximum release were calculated by counting the radioactivity in the presence of medium and 2% triton X100.

### Experimental Example 5

### Tumor Models and Vaccination with DCs

To prepare an MC-38 tumor model, MC-38 cells (2×10⁵ cells) were subcutaneously injected into C57BL/6 mice (6-8-week-old). After two days of the injection of the MC-38 cells, 1×10⁶ cells of Top2αC RNA/DC, MC38TL/DC, p1327/DC, or DC were subcutaneously injected into the mice once a week for three weeks.

To prepare an MC-38-cea2 tumor model, MC-38-cea2 cells (1×10⁶ cells) were subcutaneously injected into the C57BL/6 mice (6-8-week-old). After 10, 17, 24 days of the injection of the MC-38-cea2 cells, 1×10⁶ cells of Top2αC RNA/DC, MC-38-cea2TL/DC, or DC was subcutaneously injected into the mice.

To prepare a B16F10 tumor model, MC-38 cells (2×10⁵ cells) were subcutaneously injected into the C57BL/6 mice (6-8-week-old). After one day of the injection of the B16F10 cells, 1×10⁶ cells of Top2αC RNA/DC, MC-38-cea2TL/DC, or DC were subcutaneously injected into the mice once a week for 3 weeks.

To prepare a GL26 tumor model, GL26 cells (1×10⁶ cells) were subcutaneously injected into the C57BL/6 mice (6-8-week-old). In 7 and 14 days before the injection of the GL26 cells, 1×10⁶ cells of Top2αC RNA/DC, SuvRNA/DC, GFPRNA/DC, or DC were subcutaneously injected into the mice.

For intracranial transplantation of GL26 glioma cells, C57BL/6 mice (6 to 8-week-old) were anesthetized with ketamine/xylazine. Hairs of the mice were shaved to expose the skull. Each experimental animal was placed in a stereotactic frame using a small animal earbar. A burr hole was made using a Dremel drill approximately 3 mm lateral and 1 mm posterior from the intersection of the coronal and sagittal sutures (bregma).

GL26 cells (1×10⁴ cells) were injected to a depth of 3 mm and a volume of 4 μl using Hamilton syringes. After 4, 11 and 18 days of the injection of the GL26, 1×10⁶ cells of Top2αC RNA/DC or DC were subcutaneously vaccinated into the mice. DCs having undergone transfer without antigens were used as a negative control. Tumor growth or decreased viability was compared with that of the negative control.

Since Top2α was expressed in various tumor cell lines such as MC-38, MC-38-cea2, B16F10 and GL26, in the MC-38, MC-38-cea2, and B16F10 subcutaneous tumor models and an established GL26 intracranial and subcutaneous tumor model, the anti-tumor effect of Top2αC RNA/DC was evaluated.

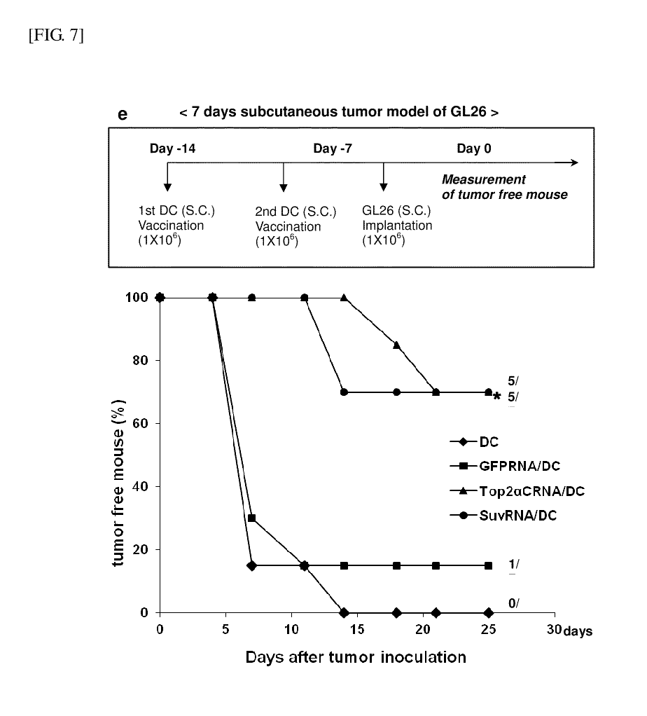

In the MC-38, MC-38-cea2, and B16F10 tumor models, the mice vaccinated with Top2αC RNA/DC, in comparison with the DC injected mice, were suppressed in tumor growth (p<0.05, FIGS. 5A, 5B, and 6C). After 4 days of the injection of GL26, the Top2αC RNA/DC-injected mice lived significantly longer than the DC-injected mice (p<0.05). 20% of the Top2αC RNA/DC-injected mice lived for 85 days or more after the tumor transplantation (FIG. 6D). In the GL26 subcutaneous tumor model, the occurrence of tumors was suppressed in 5 of 7 Top2αC RNA/DC-injected mice, while 7 DC-injected mice all had a tumor (FIG. 7).

### Experimental Example 6

### Absence of T Lymphocyte Subset In Vivo

To examine that a certain type of T lymphocyte was involved in the tumor effect in the MC-38 tumor models, the MC-38 tumor models were vaccinated with anti-CD4 monoclonal antibodies (GK1.5; eBioscience) and anti-CD8 monoclonal antibodies (2.43: a gift from Prof. Byoung S. Kwon in Ulsan University, Ulsan, Korea) on day 0, 7 and 14, and then CD4⁺ and CD8⁺ T lymphocytes were removed. FACS analysis was carried out using antibodies specific to CD4 and CD8, thereby proving that, during the treatment, CD4 and CD8 disappeared (>95%). As the control, tumor antigen-free dendritic cells were injected to vaccinate for normal tumor growth.

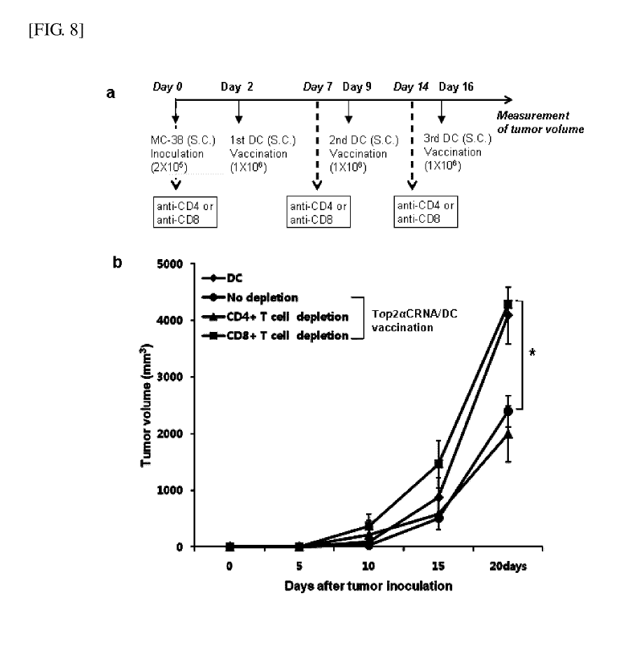

When CD8⁺ T lymphocytes were depleted, the ability to suppress tumor growth by vaccination with Top2αC RNA/DC almost completely disappeared (FIG. 8). However, when CD4⁺ T lymphocytes were depleted, there was no influence on the anti-tumor effects by Top2αC RNA/DC vaccination. These results demonstrated that CD8⁺ T lymphocytes were involved in the suppression of tumor growth in vivo by Top2αC RNA/DC vaccination.

### Experimental Example 7

### Peptide Epitopes

Top2αC-derived peptides potentially binding to H-2K^{b} were examined using a peptide motif scoring system (BIMAS; Bioinformatics and Molecular Analysis Section of NIH; http://www-bismas.cit.nih.gov. and SYFPEITHI; http://www.syfpeithi.de). A top 5 peptides having high estimated points were selected and analyzed using these two programs (Table 1). Synthetic peptides were purified at 90% minimum purity using high-performance liquid chromatography (HPLC). CEA526 (526-533: EAQNTTYL) peptides were used as negative controls.

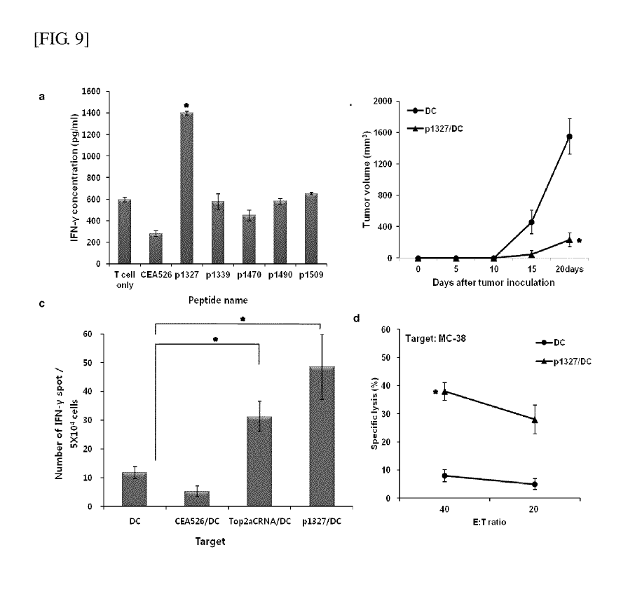

The 5 Top2αC-derived peptides binding to MHC class I molecules (H-2 Kb) with a high score were selected by BIMAS and SYFPEITHI, and in order to determine T lymphocyte epitopes of Top2αC, splenocytes from the mice vaccinated with Top2αC RNA/DCs were stimulated with Top2αC-derived peptides, and then IFN-γ production was determined by ELISA. Compared to other peptides, splenocytesactivated with P1327 (DSDEDFSGL) produced a higher level of IFN-γ (FIG. 9A). In the case of vaccination with p1327-pulsed DCs (p1327/DCs), as compared to the peptide-unloaded DCs, the tumor growth of MC-38 was significantly suppressed (FIG. 9B). To detect immune responses induced in vivo by p1327/DCs, after the third vaccination with p1327/DCs, Top2α-specific IFN-γ-secreting T lymphocytes were quantitatively analyzed using ELISPOT assay. The frequency of the IFN-γ-secreting T lymphocytes was significantly increased when being stimulated with the Top2αC RNA/DCs and p1327/DCs, in comparison with when being stimulated with the peptide-unpulsed DCs and CEA526 peptide-pulsed DCs (CEA526/DCs). The splenocytes from the mice vaccinated with the p1327/DCs exhibited cytotoxicity to the MC-38 tumor cells (FIG. 9D). As a result, it can be noted that p1327 can be one of the T lymphocyte epitopes located on the Top2αC.

TABLE 1
Top2αC-derived Peptides Expected to Bind to MHC
class I Molecule (H-2K^{b})

| | Location of | | |
|---|---|---|---|
| Name of Peptides | Peptide | Peptides Sequence | SYFPEITHI score |
| p1327 | 1327-1355 | DSDEDFSGL | 16 |
| p1339 | 1339-1447 | DEDEDFLPL | 13 |
| p1470 | 1470-1478 | SDSDFERAI | 13 |
| p1490 | 1490-1498 | EEQDFPVDL | 11 |
| p1509 | 1509-1518 | RARKPIKYL | 14 |

### Experimental Example 8

### Enzyme-Linked Immunosorbent Assay (ELISA)

Splenocytes (2×10⁵ cells/200 μl) of a mouse vaccinated with Top2αC RNA/DCs were incubated with 1 μg/ml peptides in a round-bottomed 96-well plate. Three days later, the plate was centrifuged and 100 μl of the supernatant was removed from each well. The level of IFN-γ was determined by ELISA (eBioscience, San Diego, USA).

Top2αC protein fragments, peptides or derivatives having functionally equivalent characteristics thereto induce cytotoxic immune responses specific to tumor cells, and thus can be useful as vaccines for immunotherapy of incurable cancers.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A tumor antigen comprising protein fragments, peptides derived from C-terminal of topoisomerase 2α (Top2α), or derivatives having functionally equivalent characteristics thereto and recognized by cytotoxic T lymphocytes by binding to an MHC class I or II antigen.

2. The tumor antigen according to claim 1, wherein the Top2α is derived from humans or mice.

3. The tumor antigen according to claim 1, wherein the protein fragment includes an amino acid sequence set forth in SEQ ID NO: 2 or 4.

4. The tumor antigen according to claim 1, wherein the peptides include an amino acid sequence set forth in SEQ ID NO: 5.

5. A composition for preventing or treating tumors comprising a tumor antigen of claim 1 or a gene encoding the same.

6. The composition according to claim 5, wherein the tumors express Top2α.

7. The composition according to claim 5, wherein the gene is a DNA or RNA type.

8. An antigen-presenting cells presenting a complex between an MHC class I or II antigen and a tumor antigen of claim 1 on a surface of a cell having an antigen-presenting ability.

9. The cells according to claim 8, wherein the cell having the antigen-presenting ability includes at least one selected from the group consisting of a dendritic cell, a monocyte, a CD4 T cell, a B cell, and a gamma delta (γδ) T cell.

10. The cell according to claim 9, wherein the CD4 T cell, the B cell, and the gamma delta (γδ) T cell are naive, activated or expanded.

11. An antigen-presenting cell prepared by introducing a gene encoding a tumor antigen of claim 1 into a cell having an antigen-presenting ability, and presenting a complex between an MHC class I or II antigen and the tumor antigen.

12. The cell according to claim 11, wherein the gene is a DNA or RNA type.

13. The cell according to claim 11, wherein the gene includes a base sequence set forth in SEQ ID NO: 1 or 3.

14. A composition for preventing or treating tumors comprising an antigen-presenting cell of any one of claims 8 and 10.

15. A cytotoxic T lymphocyte specifically recognizing a complex between an MHC class I or II antigen and a tumor antigen of claim 1, which is presented on an antigen-presenting cell of any one of claims 8 and 10.

16. The lymphocyte according to claim 15, which includes a CD4 or CD8 T cell.

17. A method for preparing a cytotoxic T lymphocyte, comprising stimulating an isolated lymphocyte with a tumor antigen of claim 1.

18. A composition for preventing or treating tumors comprising a cytotoxic T lymphocyte of claim 15.

19. A composition for measuring cellular immune responses comprising at least one selected from the group consisting of Top2α protein, a tumor antigen of claim 1, and an antibody specifically binding to the tumor antigen.

20. The composition according to claim 19, wherein the cellular immune responses specific for claim 1 are measured.


SEQUENCE LISTING (5 sequences)

SEQ ID NO: 1 - DNA, 1083 bp, Homo sapiens
  CDS (1)..(1080)
atgacatttattgaagaattggaggctgttgaagccaaggaaaaacaagatgaacaagtcggacttcctgggaaaggggggaaggccaaggggaaaaaaacacaaatggctgaagttttgccttctccgcgtggtcaaagagtcattccacgaataaccatagaaatgaaagcagaggcagaaaagaaaaataaaaagaaaattaagaatgaaaatactgaaggaagccctcaagaagatggtgtggaactagaaggcctaaaacaaagattagaaaagaaacagaaaagagaaccaggtacaaagacaaagaaacaaactacattggcatttaagccaatcaaaaaaggaaagaagagaaatccctggtctgattcagaatcagataggagcagtgacgaaagtaattttgatgtccctccacgagaaacagagccacggagagcagcaacaaaaacaaaattcacaatggatttggattcagatgaagatttctcagattttgatgaaaaaactgatgatgaagattttgtcccatcagatgctagtccacctaagaccaaaacttccccaaaacttagtaacaaagaactgaaaccacagaaaagtgtcgtgtcagaccttgaagctgatgatgttaagggcagtgtaccactgtcttcaagccctcctgctacacatttcccagatgaaactgaaattacaaacccagttcctaaaaagaatgtgacagtgaagaagacagcagcaaaaagtcagtcttccacctccactaccggtgccaaaaaaagggctgccccaaaaggaactaaaagggatccagctttgaattctggtgtctctcaaaagcctgatcctgccaaaaccaagaatcgccgcaaaaggaagccatccacttctgatgattctgactctaattttgagaaaattgtttcgaaagcagtcacaagcaagaaatccaagggggagagtgatgacttccatatggactttgactcagctgtggctcctcgggcaaaatctgtacgggcaaagaaacctataaagtacctggaagagtcagatgaagatgatctgttttaa

SEQ ID NO: 2 - protein, 360 aa, Homo sapiens
MTFIEELEAVEAKEKQDEQVGLPGKGGKAKGKKTQMAEVLPSPRGQRVIPRITIEMKAEAEKKNKKKIKNENTEGSPQEDGVELEGLKQRLEKKQKREPGTKTKKQTTLAFKPIKKGKKRNPWSDSESDRSSDESNFDVPPRETEPRRAATKTKFTMDLDSDEDFSDFDEKTDDEDFVPSDASPPKTKTSPKLSNKELKPQKSVVSDLEADDVKGSVPLSSSPPATHFPDETEITNPVPKKNVTVKKTAAKSQSSTSTTGAKKRAAPKGTKRDPALNSGVSQKPDPAKTKNRRKRKPSTSDDSDSNFEKIVSKAVTSKKSKGESDDFHMDFDSAVAPRAKSVRAKKPIKYLEESDEDDLF

SEQ ID NO: 3 - DNA, 801 bp, Mouse
  CDS (1)..(798)
atggtgcagaagaaagagccaggtgcaaagaagcagactacattgccgtttaagcctgtcaaaaaagggaggaagaaaaacccctggtctgactctgagtcagacgtgagcagtaatgaaagtaacgttgatgtccctcctcgacaaaaagagcaaagaagtgctgcagcaaaagccaaattcacagtggatttagactcggatgaagatttctcaggtttggatgagaaggatgaggatgaagatttcctcccattagatgctactccacctaaggccaaaatccccccaaaaaatactaaaaaagcactgaagacccagggaagctccatgtcggttgatcttgaaagtgatgttaaggacagtgtgccagcttctccaggcgttcctgctgctgacttcccagcggaaactgaacagtcaaagccatccaaaaagaccgtgggagtgaagaagacagcaaccaaaagccagtcttcagtctccactgctggtaccaaaaagagagctgcgccaaagggaaccaaatcagattcagccttgagtgctcgtgtctcggaaaaacctgctcctgccaaagccaagaacagtcgcaaaaggaagccatcttcttctgatagctctgactctgattttgagagagcaatttctaaaggtgccacgagcaagaaagcaaagggagaggaacaggacttccctgtggacttggaagacacgatagctcctcgagccaaatctgaccgggcgaggaagccaattaagtacctggaggagtccgatgacgatgacgacctcttctga

SEQ ID NO: 4 - protein, 266 aa, Mouse
MVQKKEPGAKKQTTLPFKPVKKGRKKNPWSDSESDVSSNESNVDVPPRQKEQRSAAAKAKFTVDLDSDEDFSGLDEKDEDEDFLPLDATPPKAKIPPKNTKKALKTQGSSMSVDLESDVKDSVPASPGVPAADFPAETEQSKPSKKTVGVKKTATKSQSSVSTAGTKKRAAPKGTKSDSALSARVSEKPAPAKAKNSRKRKPSSSDSSDSDFERAISKGATSKKAKGEEQDFPVDLEDTIAPRAKSDRARKPIKYLEESDDDDDLF

SEQ ID NO: 5 - protein, 9 aa, Artificial Sequence
  synthetic
DSDEDFSGL